(19) United States
(12) Patent Application Publication
(10) Pub. No.: US 2002/0160961 A1
(43) Pub. Date: Oct. 31, 2002

(54) Compositions for the treatment of the catabolic state of prolonged critical illness

(76) Inventor: Michael Ankersen, Stenlose (DK)

Correspondence Address: Reza Green, Esq., Novo Nordisk of North America, Inc., Suite 6400, 405 Lexington Avenue, New York, NY, 10174-6401, US

(21) Appl. No.: 10/004,648

(22) Filed: Dec. 4, 2001

Related U.S. Application Data
(63) Continuation of application No. PCT/DK00/00295, filed on May 31, 2000.

(30) Foreign Application Priority Data
Jun. 4, 1999 (DK) PA 1999 00788
Aug. 2, 1999 (DK) PA 1999 01082

(51) Int. Cl. A61K 38/06; A61K 38/05; C07K 5/06

(52) U.S. Cl. 514/18; 514/19; 530/331; 544/131; 544/162; 544/360; 544/386; 544/376; 546/314; 546/193; 560/38

(57) ABSTRACT

Compositions comprising TRH and a compound of the general formula A—B—C—D(—E)ₚ are used for treating the catabolic state of prolonged critical illness.

### FIELD OF INVENTION

The present invention relates to novel compositions and their use for treating the catabolic state of prolonged critical illness.

### BACKGROUND OF THE INVENTION

Growth hormone is a hormone which stimulates growth of all tissues capable of growing. In addition, growth hormone is known to have a number of effects on metabolic processes, e.g., stimulation of protein synthesis and free fatty acid mobilizaton and to cause a switch in energy metabolism from carbohydrate to fatty acid metabolism. Deficiency in growth hormone can result in a number of severe medical disorders, e.g., dwarfism.

Growth hormone is released from the pituitary The release is under tight control of a number of hormones and neurotransmitters either directly or indirectly. Growth hormone release can be stimulated by growth hormone releasing hormone (GHRH) and inhibited by somatostatin. In both cases the hormones are released from the hypothalamus but their action is mediated primarily via specific receptors located in the pituitary. Other compounds which stimulate the release of growth hormone from the pituitary have also been described For example arginine, L-3,4-dihydroxyphenylaIanine (L-Dopa), glucagon, vasopressin, PACAP (pituitary adenylyl cyclase activating peptide), muscarinic receptor agonists and a synthethic hexapeptide, GHRP (growth hormone releasing peptide) release endogenous growth hormone either by a direct effect on the pituitary or by affecting the release of GHRH and/or somatostatin from the hypothalamus.

van den Berghe et al. in “Neuroendocrinology of Prolonged Critical Illness: Effects of Exogenous Thyrotropin-Releasing Hormone and Its Combination with Growth Hormone Secretagogues”, The Journal of Clinical Endocrinology and Metabolism, Vol. 83, No. 2, February 1998, p. 309-319, describes the infusion of TRH alone or in combination with the growth hormone releasing hormone GHRP-2 for the treatment of the catabolic state of prolonged critical illness.

van den Berghe et al. describes prolonged critical illness as the phase following resuscitation of a acute life-treatening disease or trauma during which vital organ functions remain dependent on intensive care support for several weeks. In this condition, feeding is unable to reverse ongoing wasting of protein, whereas fat, in contrast, is preserved or stored.

The protein wasting results from both activated degradation and suppressed synthesis of protein. The latter determines the residual protein content, the decrease of which correlates with the duration of illness. Protein loss from vital organs and tissues aggravates dysfunction of the involved systems and, consequently, prolongs dependency on intensive care support, such as ventilation and artificial feeding.

Furthermore, van den Berghe et al. describes that when TRH was infused together with GHRP-2, it also increased pulsatile TSH secretion, thereby normalizing the TSH response

It is a further object of the present invention to provide methods, kits and uses of compounds for the treatment of the catabolic state of prolonged critical illness. It is a still further object to provide methods, kits and uses of compounds for increasing the pulsatile TSH secretion in the catabolic state of prolonged critical illness.

### SUMMARY OF THE INVENTION

Accordingly, the present invention relates to a pharmaceutical composition comprising TRH and a compound of general formula I

A—B—C—D(—E)ₚ

wherein

p is 0 or 1;

A is hydrogen or R¹—(CH₂)_{q}—(X)ᵣ—(CH₂)ₛ—CO—, wherein

q is 0 or an integer between 1 and 5;

r is 0 or 1,

s is 0 or an integer between 1 and 5;

R¹ is hydrogen, imidazolyl, guanidino, piperazino, morpholino, piperidino or N(R²)—R³,

wherein each of R² and R³ is independently hydrogen or lower alkyl optionally substituted by one or more hydroxyl, pyridinyl or furanyl groups; and

X, when r is 1, is —NH—, —CH₂—, —CH═CH—,

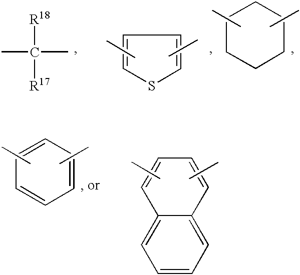

wherein each of R¹⁶ and R¹⁷ is independently hydrogen or lower alkyl;

B is (G)ₜ—(H)ᵤ wherein

t is 0 or 1;

u is 0 or1;

G and H are amino acid residues selected from the group consisting of natural L-amino acids or their corresponding D-isomers, or non-natural amino acids such as 1,4-diaminobutyric acid, aminoisobutyric acid, 1,3-diaminopropionic acid, 4-aminophenylalanine, 3-pyridylalanine, 1,2,3,4-tetrahydroisoquinoline-3-carboxylic acid, 1,2,3,4tetrahydronorharman-3-carboxylic acid, N-methylanthranilic acid, anthranilic acid, N-benzylglycine, 3-aminomethylbenzorc acid, 3-amino-3-methyl butanoic acid, sarcosine, nipecotic acid or iso-nipecotic acid;

and wherein, when both t and u are 1, the amide bond between G and H is optionally substituted by

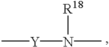

wherein Y is

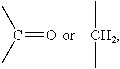

and R¹⁸ is hydrogen, lower alkyl or lower aralkyl;

C is a D-amino acid of formula —NH—CH((CH₂)_{w}—R⁴)—CO— wherein

w is 0, 1 or 2; and

R⁴ is selected from the group consisting of

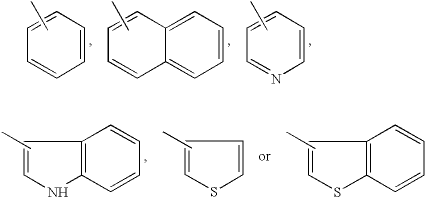

each of which is optionally substituted with halogen, lower alkyl, lower alkyloxy, lower alkylamino, amino or hydroxy;

D, when p is 1, is a D-amino acid of formula —NH—CH((CH₂)ₖ—R⁵)—CO— or, when p is 0, D is —NH—CH((CH₂)ₗ—R⁵)—CH₂—R⁶ or —NH—CH((CH₂)ₘ—R⁶)—CO—R⁸, wherein

k is 0, 1 or 2,

l is 0, 1 or 2;

m is 0, 1 or 2;

R⁶ is selected from the group consisting of

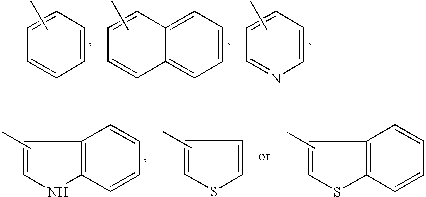

each of which is optionally substituted with halogen, alkyl, alkyloxy amino or hydroxy; and R⁶ is piperazino, morpholino, piperidino, —OH or —N(R⁷)—R⁸, wherein each of R⁷ and R⁸ is independently hydrogen or lower alkyl:

E, when p is 1, is —NH—CH(R¹⁰)—(CH₂)ᵥ—R⁹, wherein

v is 0 or an integer between 1 and 8;

R⁹ is hydrogen, imidazolyl, guanidino, piperazino, morpholino, piperidino,

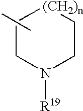

wherein n is 0, 1 or 2, and R¹⁹ is hydrogen or lower alkyl,

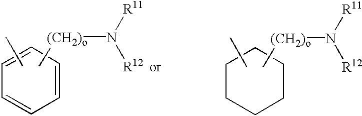

wherein o is an integer from 1 to 3,

or N(R¹¹)—R¹², wherein each of R¹¹ and R¹² is independently hydrogen or lower alkyl, or

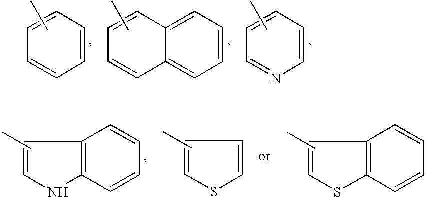

each of which is optionally substituted with halogen, alkyl, alkyloxy, amino, alkylamino, hydroxy, or the Amadori rearrangement product from an amino group and a hexapyranose or a hexapyranosyl-hexapyranose and

R¹⁰, when p is 1, is selected from the group consisting of —H, —COOH, —CH₂—R¹³, —CO—R¹³ or —CH₂—OH, wherein

R¹³ is piperazino, morpholino, piperidino, —OH or —N(R¹⁴)-R¹⁵, wherein each of R¹⁴ and R¹⁵ is independently hydrogen or lower alkyl:

the amide bond between B and C or, when t and u are both 0, between A and C being optionally substituted by

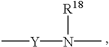

wherein Y is

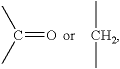

and R¹⁸ is hydrogen, lower alkyl or lower aralkyl, or, when p is 1, the amide bond between D and E being optionally substituted by

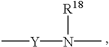

wherein Y and R¹⁸ are as indicated above;

or a pharmaceutically acceptable salt thereof, together with a pharmaceutically accacceptable carrier or diluent.

Peptide derivatives of formula I is described in WO 95/17423, which is incorporated herein by reference.

In the present context, the term “lower alkyl” is intended to indicate alkyl with 1-6 carbon atoms, in particular methyl, ethyl, propyl, iso-propyl, butyl, pentyl or hexyl. The term “halogen” is intended to include Cl, F, Br and I. In the terms “lower alkyloxy”, “lower aralkyl” and “lower alkylamino”, the lower alkyl moiety has the meaning indicated above.

### DETAILED DESCRIPTION OF THE INVENTION

In a preferred embodiment of the compound of formula I, p is 1. In another preferred embodiment of the compound of formula I, A is hydrogen or, alternatively, R¹—(CH₂)_{q}(X)ᵣ—(CH₂)ₛ—CO—, wherein R¹ is 3-imidazolyl, q is 2, r is 0 and s is 0; or wherein R¹ is NH₂, q is 1, r is 1, X is disubstituted benzene preferably substituted in the 1 and 3 positions, and s is 0; or wherein R¹ is NH₂, q is 1, r is 1, X is disubstituted thiophene preferably substituted in the 3 and 2 positions, and S is 0. In a further embodiment t is 1. When t is 1, G in the compound of formula I is preferably Ala, Gly, Aib, sarcosine, nipecotic acid or isonipecotic acid. In a still further embodiment u is 1. When u is 1, H is preferably His, Phe, Tic, Phe(4-NH₂), 3-Pyal, Gly, Ala, Sar, Pro, Tyr, Arg, Orn, 3-aminomethylbenzoic acid or D-Phe. C in the compound of formula I is preferably D-2-naphthylalanine (D-2Nal), D-1-naphthylalanine (D-1Nal), D-Phe or D-Trp. In a still further embodiment R⁴ is 2-naphthyl. D in the compound of formula I is preferably D-Phe, D-1Nal, D-2Nal, D-Trp, 3-Pyal, D-Phe(4F), D-Tyr or Phe-NH₂. In a still further embodiment R⁵ is phenyl.

E in the compound of formula I is preferably Lys-NH₂, NH-(2-(1-piperazino)ethyl), NH-(2-(1-morpholino)propyl), NH-(2-aminoethyl), NH-(4-aminomethylbenzyl), NH-(benzyl), Lys-OH, NH-(1-hydroxy-6-amino-2S-hexyl), NH-(2-(1-methyl-2-pyrrolidinyl)ehtyl), or

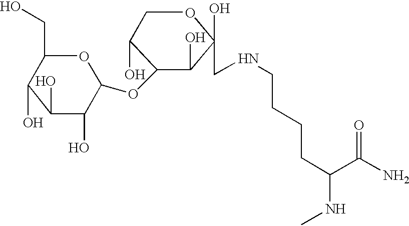

R⁴ in the compound of formula I is preferably 2-naphthyl. R⁵ is preferably phenyl, v is preferably 2-6, and R⁹ is NH₂, morpholinoethyl, morpholinopropyl or (1-methylpyrrolidinyl)ethyl. R¹⁰ is preferably —COOH, —CH₂—OH, —H, —CONH₂ or —CON(CH₃)₂.

In a special embodiment of the compound of formula I

p is 1;

A is hydrogen or R¹—(CH₂)_{q}—(X)ᵣ—(CH₂)ₛ—CO—,

wherein R¹ is 3-imidazolyl, q is 2, r is 0 and s is 0;

or wherein R¹ is NH₂, q is 1, r is 1, X is disubsttuted benzene preferably substituted in the 1 and 3 positions, and s is 0;

or wherein R¹ is NH, q is 1, r is 1, X is disubstituted thiophene preferably substituted in the 3 and 2 positions, and s is 0;

t is 1;

G is Ala, Gly, Aib, sarcosine, nipecotic acid, or iso-nipecotic acid;

u is 1;

H is His, Phe, Tic, 3Pyal, Gly, Ala, Phe(4-NH₂), Sar, Pro, Tyr, Arg, Orn, 3-aminomethylbenzoic acid or D-Phe;

R⁴ is 2-naphthyl;

R⁵ is phenyl;

v is 2, 3, 4, 5, or 6;

R⁹ is —NH₂, morpholinopropyl, morphoninoethyl or (1-methylpyrrolidinyl)ethyl; and

R¹⁰ is —COOH, —CH₂—OH, —H or —CONH₂.

Any possible combination of two or more of the embodiments described herein is comprised within the scope of the invention,

Examples of specific compounds of formula I are

H-Ala-Hisψ(CH₂NH)D-2Nal-D-Phe-Lys-NH₂

H-Ala-Ala-D-2Nal-D-Phe-Lys-NH₂

H-His-D-2Nal-D-Phe-Lys-NH₂

(3-(4-imidazolyl)propionyl)-D-2Nal-D-Phe-Lys-NH₂

H-D-Lys-D-2Nal-Phe-Lys-NH₂

H-5Apent-His-D-2Nal-D-Phe-Lys-NH₂

H-D-Ala-D-2Nal-D-Phe-Lys-NH₂

H-5Apent-D-2Nal-D-Phe-Lys-NH₂

(n-Propyl)-His-D-2Nal-D-Phe-Lys-NH₂

H-Ala-3Pyal-D-2Nal-D-Phe-Lys-NH₂

H-Ala-Phe(4NH₂)-D-2Nal-D-Phe-Lys-NH₂

H-D-Ala-His-D-2Nal-D-Phe-Lys-NH₂

(2-(4-imidazolyl)acetyl)-D-2Nal-D-Phe-Lys-NH₂

(3-(4-imidazolyl)acryloyl)-D-2Nal-D-Phe-Lys-NH₂

(3-Aminomethylbenzoyl)-D-2Nal-D-Phe-Lys-NH₂

(3-Aminophenylacetyl)-D-2Nal-D-Phe-Lys-NH₂

(4-Aminophenylacetyl)-D-2Nal-D-Phe-Lys-NH₂

(3-Aminocrotonoyl)-D-2Nal-D-Phe-Lys-NH₂

(4-Piperidino-carboxyl)-D-2Nal-D-Phe-Lys-NH₂

H-Ala-His-D-2Nal-D-Phe-NH₂

(H-Ala-His-D-2Nal-D-Phe-NH)hexane

6-(H-Ala-His-D-2Nal-D-Phe-NH)hexylamine

5-(H-Ala-His-D-2Nal-D-Phe-NH)pentylanaine

H-Ala-His-D-2Nal-D-Pheψ(CH₂NH)Lys-NH₂

H-Ala-His-D-2Nal-D-Phe-Lys-OH

(2S)(H-Ala-His-2Nal-D-Phe-NH)-6-aminohexanol

(2-(H-Ala-His-D-2Nal-D-Phe-NH)ethyl)benzene

2-(H-Ala-His-D-2Nal-D-Phe-NH)ethylamine

4-((H-Ala-His-D-2Nal-D-Phe-NH)methyl)benzylamine

H-Ala-His-D-2Nal-D-Phe-Lys(maltosyl)-NH₂

H-Ala-His-D-2Nal-D-Phe-Phe-NH₂

H-Ala-His-D-2Nal-D-Phe-D-Phe-NH₂

H-Ala-His-D-Phe-D-Phe-Lys-NH₂

H-Ala-His-D-Trp-D-Phe-Lys-NH₂

H-His-D-2Nal-D-Trp-Lys-NH₂

H-Ala-His-D-1Nal-D-Phe-Lys-NH₂

H-Ala-Phe-D-2Nal-D-Phe-Lys-NH₂

H-Ala-His-D-2Nal-D-Phe-Lys(maltosyl)-NH₂

(2R)-(H-Ala-His-D-2Nal-D-Phe-Lys-NH)-3phenylpropylamine

H-Ala-N-Me(2-aminobenzoyl)-1D-2Nal-D-Phe-Lys-NH₂

(3-(Methylaminomethyl)benzoyl)-D-2Nal-D-Phe-Lys-NH₂

(4-(Aminomethyl)benzoyl)-D-2Nal-D-Phe-Lys-NH₂

H-His-Ala-D-2Nal-D-Phe-Lys-NH₂

4-(H-Ala-His-D-2Nal-D-Phe-NH)butylamine

3-(H-Ala-His-D-2Nal-D-Phe-NH)propylamine

(3-(Dimethylaminomethyl)benzoyl)-D-2Nal-D-Phe-Lys-NH₂

(3-Amino-3-methylbutanoyl)-D-2Nal-D-Phe-Lys-NH₂

(3-Aminomethylbenzoyl)-D-hPhe-D-Phe-Lys-NH₂

(3-Aminomethylbenzoyl)ψ(CH₂NH)-D-2Nal-D-Phe-Lys-NH₂

(3-Aminomethylbenzoyl)-D-2Nal-D-hPhe-Lys-NH₂

(3-Amino-3-methylbutanoyl)-His-D-2Nal-D-Phe-Lys-NH₂

(3-Aminomethylbenzoyl)-3-2Nal-N-Bzl-Gly-Lys-NHz

(2S)-(3-aminomethylbenzoyl)ψ(CH₂NH)-D-2Nal-D-Phe-NH)-6-aminohexanol

(2S)-((3-aminomethylbenzoyl)-D-2Nal-D-Phe-NH)-6-aminohexanol

(3-Aminomethylbenzoyl)-D-2Nal-D-Thial-Lys-NH₂

(2S)-(H-Aib-Hisψ(CH₂NH)-D-2Nal-D-Phe-NH)-6-aminohexanol

(3-Aminomethylbenzoyl)-D-2Nal-D-3Pyal-Lys-NH₂

(3-Aminomethylbenzoyl)-D-2Nal-D-Phe(4-F)-Lys-NH₂

(3-Aminomethylbenzoyl)-D-2Nal-D-Phe(4-OMe)-Lys-NH₂

(2-Aminomethylphenylacetyl)-D-2Nal-D-Phe-Lys-NH₂

(2-Aminomethylbenzoyl)-D-2Nal-D-Phe-Lys-NH₂

2-(H-Aib-His-D2Nal-D-Phe-NH)-(4pyridyl)ethane

H-Aib-Phe-D-2Nal-D-Phe-Lys-NH₂

2-(H-Aib-His-D-2Nal-D-Phe-NH)-(1-methyl-2-pyrrolidinyl)ethane

2-(H-Aib-His-D-2Nal-(-D-Phe-NH)-(4-pyridyl)ethane

H-Aib-Hisψ(CH₂NH)-D-2Nal-D-Phe-Lys-OH

(3-Aminomethylbenzoyl)-D-2Nal-N-Me-D-Phe-Lys-NH₂

H-Aib-His-D-2Nal-D-Phe-Gly-NH₂

H-Aib-His-D-2Nal-D-Phe-Ala-NH₂

H-Aib-His-D-2Nal-D-Phe-Orn-NH₂

(5-Aminomethylthienyl-2-carbonyl)-D-2Nal-D-Phe-Lys-NH₂

H-Aib-His-D-2Nal-D-PhD-Lys-NH₂

H-Aib-His-D-2Nal-D-Phe-Dab-NH₂

H-Aib-His-D-2Nal-D-Pheψ(CH₂NH)-Lys-NH₂

H-Aib-His-N-Me-D-2Nal-D-Phe-Lys-NH₂

H-Aib-His-D-2Nal-D-Phe-N-Me-Lys-NH₂

(3-Aminomethylthienyl-2-carbonyl)-D-2Nal-D-Phe-Lys-NH₂

H-Aib-His-D-2Nal-N-Me-D-Phe-Lys-NH₂

H-Aib-His-D-2Nal-D-Phe-Lys-N(Me)₂

(3R)-Pipendinecarbonyl-D-2Nal-D-Phe-Lys-NH₂

(3S)-Pipendinecarbonyl-D-2Nal-D-Phe-Lys-NH₂

(3-Aminomethylbenzoyl)-D-1Nal-D-Phe-Lys-NH₂

H-Aib-His-D-2Nal-D-Trp-Lys-NH₂

(Furfuryl-Aib-His-D-2Nal-D-Phe-Lys-NH₂

(2-Pyridyl(methyl)-Aib-His-D-2Nal-D-Phe-Lys-NH₂

H-Aib-(3-aminomethylbenzoyl)-D-2Nal-D-Phe-Lys-NH₂

H-Aib-3Pyal-D-2Nal-D-Phe-Lys-NH₂

(3S)-Piperidinecarbonyl-D-2Nal-D-Phe-Lys-NH₂

(3R)-Pipendinecarbonyl-D-2Nal-D-Phe-Lys-NH₂

(2-(H-Aib-His-D-2Nal-NH)ethyl)benzene

N,N-di(2R-Hydroxypropyl)-(3-aminomethylbenzoyl)-D-2Nal-D-Phe-Lys-NH₂

(2R-Hydroxypropyl)-Aib-His-D-2Nal-D-Phe-Lys-NH₂

(3-Aminomethylbenzoyl)-D-2Nal-D-Pheψ(CH₂NH)Lys-NH₂

(3-Aminomethylbenzoyl)-N-Me-D-2Nal-D-Phe-Lys-NH₂

(3-Aminomethylbenzoyl)-D-2Nal-D-Phe-N-Me-Lys-NH₂

H-D-Thr-His-D-2Nal-D-Phe-Lys-NH₂

H-Aib-His-D-2Nal-N-(phenethyl)Gly-Lys-NH₂

(3Aminomethylbenzoyl)-D-2Nal-N-(phenethyl)-Gly-Lys-NH₂

H-Hyp-His-D-2Nal-D-Phe-Lys-NH₂

H-Aib-His-N-Me-D-2Nal-N-(phenethyl)-Gly-Lys-NH₂

H-Aib-His-N-Me-D2Nal-N-Me-D-Phe-Lys-NH₂

H-Aib-His-D-2Nal-D-Pheψ(CH₂N(Me))Lys-NH₂

3(H-Aib-His-D-2Nal-N-Me-D-Phe-NH)morpholinopropane

2-(H-Aib-His-D-2Nal-N-Me-D-Phe-NH)-(1-methyl-2-pyrrolidinyl)ethane

(3R)-Piperidinecarbonyl—N-Me-D-2Nal-N-Me-D-Phe-Lys-NH₂

3-((Aminomethylbenzoyl)-D-2Nal-N-Me-D-Phe-NH)morpholinopropane

2-(H-Aib-His-N-Me-D-2Nal-N-Me-D-Phe-NH)-(1-methyl-2-pyrrolidinyl)ethane

2-(3R)-Piperidinecarbonyl-N-Me-D-2Nal-N-Me-D-Phe-NH)-(1-methyl-2-pyrrolidinyl)ethane

2-(3-Aminomethylbenzoyl)-N-Me-D-2Nal-N-Me-D-Phe-NH)-(1-methyl-2-pyrrolidinyl)ethane

3-(H-Aib-His-N-Me-D-2Nal-N-Me-D-Phe-NH)morpholinopropane

3-((3R)-Piperdinecarbonyl-N-Me-D-2Nal-N-Me-D-Phe-NH)morpholinopropane

3-((3-Aminomethylbenzoyl)-N-Me-D-2Nal-N-Me-D-Phe-NH)morpholinopropane

H-Aib-His-D-2Nal-N-Me-D-Phe-Hyp-NH₂

2-((3-Aminomethylbenzoyl)-D-2Nal-N-Me-D-Phe-NH)-(1-methyl-2-pyrrolidinylethane

2-((3R)Piperidinecarbonyl-D-2Nal-N-Me-D-Phe-NH)-(1-methyl-2-pyrrolidinyl)ethane

Preferred compounds of formula I of the invention are:

ipamorelin (H-Aib-His-D-2Nal-D-Phe-Lys-NH₂)

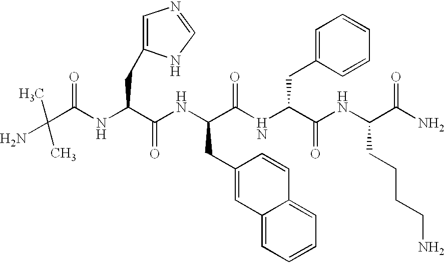

and pharmaceutically acceptable salts thereof.

Abbreviations:

D-2Nal=D-2-naphthytalanine

5Apent=5-aminopentanoic acid

3Pyal=3-pyridylalanine

Aib=H-amino-isobutync acid

Thial=thienylaianine

hPhe=homo-phenylalanine

N-Bzl-Gly=N-benzylglycine

4-F=4-fluoro

4-OMe=4-methoxy

Orn=ornithine

Dab=2,4-diaminobutyric acid

Hyp=hydroxyproline

Tic=1,2,3,4-tetrahydroisoquinoline-3-carboxylic acid

Compounds of formula I may be prepared by conventional methods of solution or solid phase peptide synthesis. For instance, solid phase synthesis may be carried out substantially as described by Stewart and Young, Solid Phase Peptide Synthesis, 2nd. Ed., Rockford, Ill., USA, 1976. Solution peptide synthesis may for instance be carried out substantially as described by Bodansky et al., Peptide Synthesis, 2nd. Ed., New York, N.Y., USA, 1976.

Aminomethylene as a substitution of an amide bond may be introduced according to the method described by Y. Sasaki and D. H. Coy. Peptides 8(1), 1987, pp. 119-121. Peptide derivatives containing a mono- or di-hexapyranose derivatised amino group may be prepared by an Amadori rearrangement substantially be the method described by R. Albert et al., Life Sciences 53, 1993, pp. 517-525. Examples of suitable mono- or di-hexapyranoses are glucose, galactose, maltose, lactose or cellobiose. Derivatives used as starting materials in the synthesis may either be obtained commercially and, when required, provided with suitable protecting groups, or starting materials used to prepare the “A” moiety in general formula I may be prepared by well-known methods and optionally protected in a manner known per se.

Pharmaceutically acceptable acid addition salts of compounds of formula I include those prepared by reacting the peptide with an inorganic or organic acid such as hydrochloric, hydrobromic, sulfuric, acetic, phosphoric, lactic, maleic, phthalic, citric, glutaric, gluconic, methanesulfonic, salicylic, succinic, tartaric, toluenesulfonic, trifluoracetic, sulfamic and fumaric acid.

In another aspect, the present invention relates to a pharmaceutical composition comprising, as an active ingredient, a compound of the general formula I or a pharmaceutically acceptable salt thereof together with a pharmaceutically acceptable carrier or diluent.

Pharmaceutical compositions containing a compound of the present invention may be prepared by conventional techniques, e.g. as described in Remington's Pharmaceutical Sciences, 1985. The compositions may appear in conventional forms, for example capsules, tablets, aerosols, solutions, suspensions or topical applications.

The pharmaceutical carrier or diluent employed may be a conventional solid or liquid carrier Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene and water.

Similarly, the carrier or diluent may include any sustained release material known in the art, such as glyceryl monostearate or glyceryl distearate, alone or mixed with a wax.

If a solid carrier is used for oral administration, the preparation may be tabletted, placed in a hard gelatin capsule in powder or pellet form or it can be in the form of a troche or lozenge. The amount of solid carrier will vary widely but will usually be from about 25 mg to about 1 g.

A typical tablet which may be prepared by conventional tabletting techniques may contain,

| Core: | | |
|---|---|---|
| Active compound (as free compound or salt thereof) | 100 | mg |
| Colloidal silicon dioxide (Aerosil) | 1.5 | mg |
| Cellulose, microcryst. (Avicel) | 70 | mg |
| Modified cellulose gum (Ac-Di-Sol) | 7.5 | mg |
| Magnesium stearate | | |
| Coating: | | |
| HPMC approx. | 9 | mg |
| *Mywacett 9-40 T approx. | 0.9 | mg |

*Acylated monoglyceride used as plasticizer for film coating.

If a liquid carrier is used, the preparation may be in the form of a syrup, emulsion, soft gelatin capsule or sterile injectable liquid such as an aqueous or non-aqueous liquid suspension or solution.

For nasal administration, the preparation may contain a compound of formula I dissolved or suspended in a liquid carrier, in particular an aqueous carrier, for aerosol application. The carrier may contain additives such as solubilizing agents, e.g. propylene glycol, surfactants such as bile acid salts or polyoxyethylene higher alcohol ethers, absorption enhancers such as lecithin (phosphatidylcholine) or cyclodextrin, or preservatives such as parabenes.

Generally, the compounds of the present invention are dispensed in unit dosage form comprising 0.0001-100 mg of active ingredient together with a pharmaceutically acceptable carrier per unit dosage.

The dosage of the compounds according to this invention is suitably 1-500 mg/day, e.g. about 100 mg per dose, when administered to patients, e.g. humans, as a drug.

It has been demonstrated that compounds of the general formula I possess the ability to release endogenous growth hormone in vivo. The compounds may therefore be used in the treatment of conditions which require increased plasma growth hormone levels such as in growth hormone deficient humans or in elderly patients or livestock.

Thus, in a particular aspect, the present invention relates to a pharrnaceutical composition for stimulating the release of growth hormone from the pituitary, the composition comprising, as an active ingredient, a compound of the general formula I or a pharmaceutically acceptable salt thereof together with a pharmaceutically acceptable carrier or diluent.

In a further aspect, the present invention relates to a method of stimulating the release of growth hormone from the pituitary, the method comprising administering to a subject in need thereof an effective amount of a compound of the general formula I or a pharmaceutically acceptable salt thereof.

In a still further aspect the present invention relates to a pharmaceutical composition comprising a compound of the general formula I or a pharmaceutically acceptable salt thereof and TRH, together with a pharmaceutically acceptable carrier or diluent. In a preferred embodiment the compound of the general formula I is ipamorelin or a pharmaceutically acceptable salt thereof.

In a further aspect the present invention relates to a pharmaceutical composition comprising a compound of the general formula I or a pharmaceutically acceptable salt thereof and a TRH analogue, together with a pharmaceutically acceptable carrier or diluent. In a preferred embodiment the compound of the general formula I is ipamorelin or a pharmaceutically acceptable salt thereof.

In the present context, the term “TRH” is intended to mean thyrotropin-releasing hormone. TRH is inter alia capable of stimulating the secretion of thyroid stimulating hormone (TSH).

In the present context, the term “a TRH analogue” is intended to mean any compound that stimulates the secretion of thyroid stimulating hormone (TSH), including prodrugs and metabolites of such compounds and of TRH, preferably with essentially the same TSH release as TRH.

Examples of TRH analogues are e.g NS-3 (montirelin hydrate), CNK-602A, taltirelin hydrate (TA-0910), dimethyl proline-TRH (RX77368), pGlu-3-methyl His2-Pro amide (M-TRH), Z-p-Glu-His-Pro-NH₂, 1p-Glu-Tyr-Pro-NH₁, and posatirelin (L-6-ketopiperidine-2-carbonyl-L-leucyl-proline amide, RGH-2202).

In a further aspect the present invention relates to a kit comprising a compound of the general formula I or a pharmaceutically acceptable salt thereof and TRH In one embodiment the invention relates to a kit comprising ipamorelin or a pharmaceutically acceptable salt thereof and TRH.

In a still further aspect the present invention relates to a kit comprising a compound of the general formula I or a pharmaceutically acceptable salt thereof and a TRH analogue. In one embodiment the invention relates to a kit comprising ipamorelin or a pharmaceutically acceptable salt thereof and a TRH analogue.

In a further aspect the present invention relates to a method for the treatment of the catabolic state of prolonged critical illness, the method comprising administering to a subject in need thereof an effective amount of a compound of the general formula I or a pharmaceutically acceptable salt thereof and an effective amount of TRH. In one embodiment the compound of the general formula I is ipamorelin or a pharmaceutically acceptable salt thereof. In a special embodiment the effective amount of TRH is approximately the same amount as described by van den Berghe et al. (in “Neuroendocrinology of Prolonged Critical Illness: Effects of Exogenous Thyrotropin-Releasing Hormone and Its Combination with Growth Hormone Secretagogues”, The Journal of Clinical Endocrinology and Metabolism, Vol. 83, No. 2, February 1998, p, 309-319) and the effective amount of ipamorelin is the amount which gives approximately the same effect as the amount of GHRP-2 as described by van den 13erghe et al In a more special embodiment the effective amount of ipamorelin is approximately 2-3 times the amount of GHRP-2 as described by van den Berghe et al.

In a still further aspect the present invention relates to a method for the treatment of the catabolic state of prolonged critical illness, the method comprising administering to a subject in need thereof an effective amount of a compound of the general formula I or a pharmaceutically acceptable salt thereof and an effective amount of a TRH analogue. In one embodiment the compound of the general formula I is ipamorelin or a pharmaceutically acceptable salt thereof. In a special embodiment the effective amount of the TRH analogue is the amount which gives approximately the same effect as the amount of TRH as described by van den Berghe et al. (in “Neuroendocrinology of Prolonged Critical Illness. Effects of Exogenous Thyrotropin-Releasing Hormone and Its Combination with Growth Hormone Secretagogues”, The Journal of Clinical Endocrinology and Metabolism, Vol. 83, No. 2, February 1998, p. 309-319) and the effective amount of ipamorelin is the amount which gives approximately the same effect as the amount of GHRP-2 as described by van den Berghe et al. In a more special embodiment the effective amount of ipamorelin is approximately 2-3 times the amount of GHRP-2 as described by van den Berghe et al.

The effective amounts mentioned above are the amounts of a compound of the general formula I or a pharmaceutically acceptable salt thereof and of TRH or of a TRH analogue which amounts in combination are effective in the treatment of the catabolic state of prolonged critical illness.

In a further aspect, the present invention relates to the use of a compound of the general formula I or a pharmaceutically acceptable salt thereof for the preparation of a medicament for stimulating the release of growth hormone from the pituitary.

To those skilled in the art, it is well known that the current and potential uses of growth hormone in humans are varied and multitudinous. Thus, compounds of formula I can be administered for purposes stimulating release of growth hormone from the pituitary and would then have similar effects or uses as growth hormone itself. Compounds of formula I are useful for stimulation of growth hormone release in the elderly; prevention of catabolic side effects of glucocorticoids, prevention and treatment of osteoporosis, treatment of chronic fatigue syndrom (CFS), treatment of acute fatigue syndrom and muscle loss following election surgery, stimulation of the immune system, acceleration of wound healing accelerating bone fracture repair, accelerating complicated fractures, e.g disctraction osteogenesis, treatment of wasting secondary to fractures, treatment of growth retardation, treatment of growth retardation resulting from renal failure or insufficiency, treatment of cardiomyopathy, treatment of wasting in connection with chronic liver disease, treatment of thrombocytopenia, treatment of growth retardation in connection with Crohn's disease, treatment of short bowel syndrome, treatment of wasting in connection with chronic obstructive pulmonary disease (COPD), treatment of complications associated with transplantation, treatment of physiological short stature including growth hormone deficient children and short stature associated with chronic illness. treatment of obesity and growth retardation associated with obesity, treatment of anorexia, treatment of growth retardation associated with the Prader-Willi syndrome and Tumer's syndrome; increasing the growth rate of a patient having partial growth hormone insensitive syndrome, accelerating the recovery and reducing hospitalization of burn patients; treatment of intrauterine growth retardation, skeletal dysplasia, hypercortisolism and Cushing's syndrome; induction of pulsatile growth hormone release; replacement of growth hormone in stressed patients, treatment of osteochondrodysplasias, Noonan's syndrome, schizophrenia, depressions, Alzheimer's disease, delayed wound healing and psychosocial deprivation, treatment of catabolism in connection with pulmonary dysfunction and ventilator dependency, treatment of cardiac failure or related vascular dysfunction, treatment of impaired cardiac function, treatment or prevention of myocardial infarction, lowering blood pressure, protection against ventricular dysfunction or prevention of reperfusion events, treatment of adults in chronic dialysis, attenuation of protein catabolic responses after major surgery, reducing cachexia and protein loss due to chronic illness such as cancer or AIDS: treatment of hyperinsulinemia including nesidioblastosis, adjuvant treatment for ovulation induction; stimulation of thymic development and prevention of the age-related decline of thymic function, treatment of immunosuppressed patients, treatment of sarcopenia, treatment of wasting in connection with AIDS, improvement in muscle strength, mobility, maintenance of skin thickness, treatment of metabolic homeostasis and renal homeostasis in the frail elderly, stimulation of osteoblasts, bone remodelling and cartilage growth, regulation of food intake, stimulation of the immune system in companion animals and treatment of disorders of aging in companion animals, promoting growth in livestock and stimulation of wool growth in sheep, increasing milk production in livestock, treatment of metabolic syndrome (syndrome X), treatment of insulin resistance, including NIDDM, in mammals, e.g. humans, treatment of insulin resistance in the heart, improvement of sleep quality and correction of the relative hyposomatotropism of senescence due to high increase in REM sleep and a decrease in REM latency, treatment of hypothermia, treatment of frailty associated with aging, treatment of congestive heart failure, treatment of hip fractures, treatment of immune deficiency in individuals with a depressed T4/T8 cell ratio, treatment of muscular atrophy, treatment of musculoskeletal impairment in elderly, enhancing the activity of protein kinase B (PKB), improvement of the overall pulmonary function, treatment of sleep disorders, and the treatment of the catabolic state of prolonged critical illness. Treatment is also intended to include prophylactic treatment.

In one embodiment the compounds of formula I can be used for the treatment of the catabolic state of prolonged critical illness. In a preferred embodiment the compound ipamorelin or a pharmaceutically acceptable salt thereof can be used for the treatment of the catabolic state of prolonged critical illness.

For the above indications the dosage may vary depending on the compound of formula I employed, on the mode of administration and on the therapy desired. However, generally dosage levels between 0.0001 and 100 mg/kg body weight per day may be administered to patients and animals to obtain effective release of endogenous growth hormone. Usually, dosage forms suitable for oral or nasal administration comprise from about 0.0001 mg to about 100 mg, preferably from about 0.001 mg to about 50 mg of the compounds of formula I admixed with a pharmaceutically acceptable carrier or diluent.

The compounds of formula I may be administered in pharmaceutically acceptable acid addition salt form or, where appropriate, as a alkali metal or alkaline earth metal or lower alkylammonium salt. Such salt forms are believed to exhibit approximately the same order of activity as the free base forms.

Optionally, the pharmaceutical composition of the invention may comprise a compound of formula I combined with one or more compounds exhibiting a different activity, e.g., an antibiotic or other pharmacologically active material. This might be another secretagogue, such as GHRP (1 or 6) or GHRH or an analogue thereof, growth hormone or an analogue thereof or a somatomedin such as IGF-1 or IGF-2.

In one embodiment the compounds of formula I can be administered together with one or more compounds exhibiting a different activity, e.g. an anabolic effect. In a special embodiment the compounds of formula I can be administered together with TRH, a TRH analogue, insulin or a compound having an anabolic effect.

In a preferred embodiment the compound ipamorelin can be administered together with TRH or a TRH analogue. In a still further embodiment ipamorelin can be administered together with TRH. In a further embodiment ipamorelin can be administered together with a TRH analogue.

In a still further aspect the present invention relates to the use of a compound of the general formula I or a pharmaceutically acceptable salt thereof together with TRH for the preparation of a medicament for the treatment of the catabolic state of prolonged critical illness. In one embodiment the compound according to the general formula I is ipamorelin or a pharmaceutically acceptabel salt thereof.

In a further aspect present invention relates to the use of a compound of the general formula I or a pharmaceutically acceptable salt thereof together with a TRH analogue for the preparation of a medicament for the treatment of the catabolic state of prolonged critical illness. In one embodiment the compound according to the general formula I is ipamorelin or a pharmaceutically acceptabel salt thereof.

In a still further aspect the present invention relates to the use of ipamorelin or a pharmaceutically acceptable salt thereof for the preparation of a medicament for use in the treatment of the catabolic state of prolonged critical illness in a regimen which additionally comprises treatment with TRH.

In a further aspect the present invention relates to the use of ipamorelin or a pharmaceutically acceptable salt thereof for the preparation of a medicament for use in the treatment of the catabolic state of prolonged critical illness in a regimen which additionally comprises treatment with a TRH analogue.

The route of administration may be any route which effectively transports the active compound to the appropriate or desired site of action, such as oral, nasal or parenteral, the oral route being preferred.

In one embodiment the route of administration in connection with treatment of the catabolic state of prolonged critical illness is parenteral administration. In a second embodiment the administration of a compound of the general formula I or a pharmaceutically acceptable salt thereof together and TRH or a TRH analogue takes place as continuous infusion of both compounds. In a third embodiment the administration of a compound of the general formula I or a pharmaceutically acceptable salt thereof together and TRH or a TRH analogue takes place separately.

The invention is further illustrated in the following examples which are not in any way intended to limit the scope of the invention as claimed.

The abbreviations used throughout this description and examples indicate the following structures.

Abbreviations for non-natural amino acid residues:

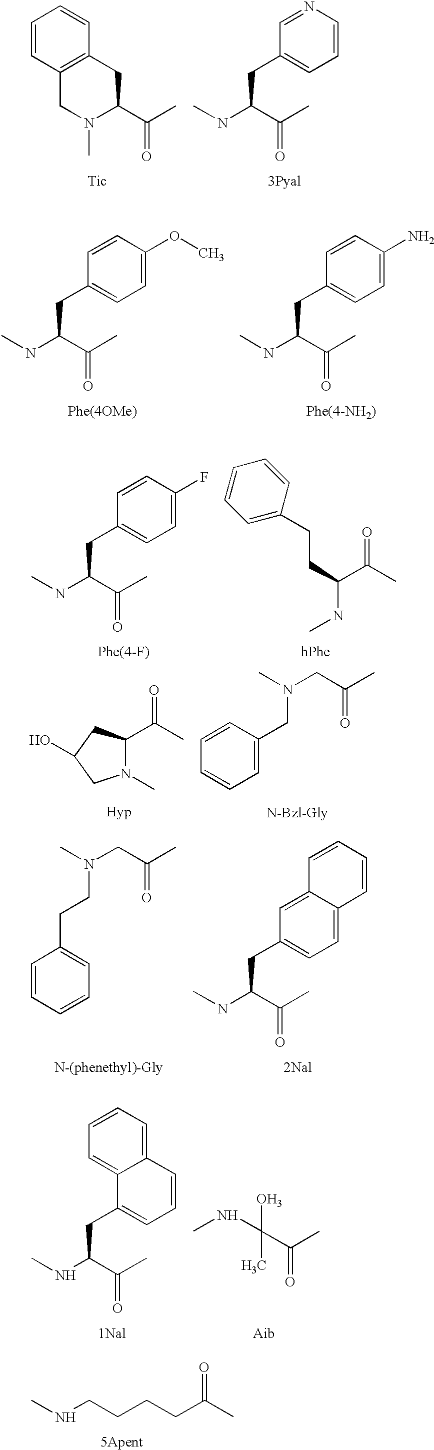

Abbreviations used for peptide bond substitutions:

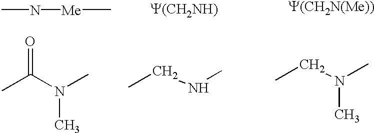

Abbreviations used for protecting groups:

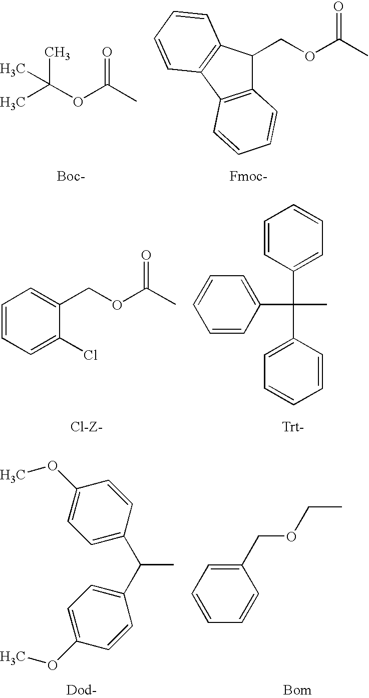

### EXAMPLES

The compounds of formula I may be prepared and evaluated for their efficacy and potency to release growth hormone as described in WO 95/17423, which is incorporated herein by reference.

1. A pharmaceutical composition comprising TRH and a compound of general formula I
A—B—C—D(—E)ₚ
wherein
p is 0 or 1;
A is hydrogen or R¹—(CH₂)_{q}—(X)ᵣ—(CH₂)ₛ—CO—, wherein
q is 0 or an integer between 1 and 5;
r is 0 or 1
s is 0 or an integer between 1 and 5;
R¹ is hydrogen, imidazolyl, guanidino, piperazino, morpholino, piperidino or N(R²)—R³,
wherein each of R² and R³ is independently hydrogen or lower alkyl optionally substituted by one or more hydroxyl, pyridinyl or furanyl groups; and
X, when r is 1, is —NH—, —CH₂—, —CH═CH—, 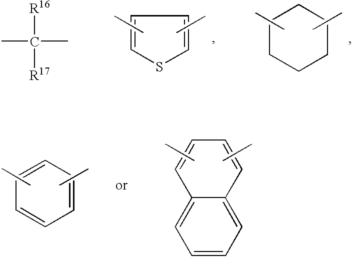
wherein each of R¹⁶ and R¹⁷ is independently hydrogen or lower alkyl
B is (G)ₜ—(H)ᵤ wherein
t is 0 or 1;
u is 0 or 1;
G and H are amino acid residues selected from the group consisting of natural L-amino acids or their corresponding D-isomers, or non-natural amino acids such as 1,4-diaminobutyric acid, amino-isobutyri acid, 1,3-diaminopropionic acid, 4-aminophenylalanine, 3-pyridylalanine, 1,2,3,4-tetrahydroisoquinoline-3-carboxylic acid, 1,2,3,4-tetrahydronorharman-3-carboxylic acid, N-methylanthranilic acid, anthranilic acid, N-benzylglycine, 3-amino-3-methylbenzoic acid, 3-amino-3-methyl butanoic acid, sarcosine, nipecotic acid or iso-nipecotic acid;
and wherein, when both t and u are 1, the amide bond between G and H is optionally substituted by 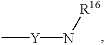
wherein Y is 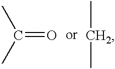
and R¹⁸ is hydrogen, lower alkyl or lower aralkyl;
C is a D-amino acid of formula —NH—CH((CH₂)_{w}—R⁴)—CO— wherein
w is 0, 1 or 2; and
R⁴ is selected from the group consisting of 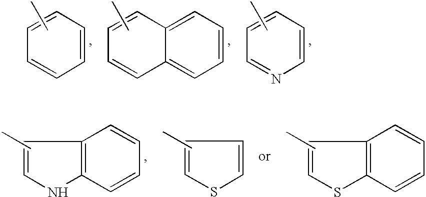
each of which is optionally substituted with halogen, lower alkyl, lower alkyloxy, lower alkylamino, amino or hydroxy;
D, when p is 1, is a D-amino acid of formula —NH—CH((CH₂)ₖ—R⁵)—CO— or, when p is 0, D is —NH—CH((CH₂)ᵣ—R⁵)—CH₂—R⁶ or —NH—CH((CH₂)ₘ—R⁵)—CO—R⁶, wherein
k is 0, 1 or 2;
l is 0, 1 or 2;
m is 0, 1 or 2;
R⁵ is selected from the group consisting of 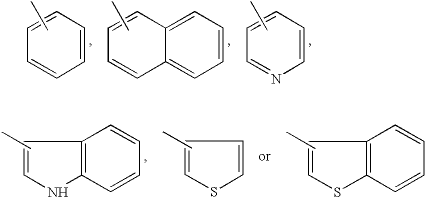
each of which is optionally substituted with halogen, alkyl, alkyloxy amino or hydroxy; and R⁵ is piperazino, morpholino, piperidino, —OH or —N(R⁷)—R⁸, wherein each of R⁷ and R⁸ is independently hydrogen or lower alkyl;
E, when p is 1, is —NH—CH(R¹⁰)—(CH₂)ᵥ—R⁹, wherein
v is 0 or an integer between 1 and 8;
R⁹ is hydrogen, imidazolyl, guanidino, piperazino, morpholino, piperidino, 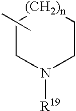
wherein n is 0, 1 or 2, and R¹⁹ is hydrogen or lower alky, 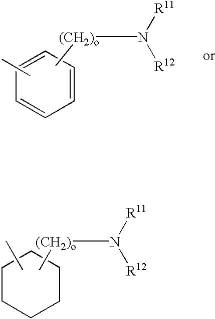
wherein o is an integer from 1 to 3,
or N(R¹¹)—R¹², wherein each of R¹¹ and R¹² is independently hydrogen or lower alkyl, or 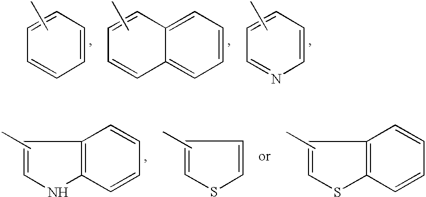
each of which is optionally substituted with halogen, alkyl, alkyloxy, amino, alkylamino, hydroxy, or the Amadori rearrangement product from an amino group and a hexapyranose or a hexapyranosyl-hexapyranose and
R¹⁰, when p is 1, is selected from the group consisting of —H, —COOH, —CH₂—R¹³, —CO—R¹³ or —CH₂—OH, wherein
R¹³ is piperazino, morpholino, piperidino, —OH or —N(R¹⁴)—R¹⁵, wherein each of R¹⁴ and R¹⁵ is independently hydrogen or lower alkyl;
the amide bond between B and C or, when t and u are both 0, between A and C being optionally substituted by 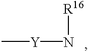
wherein Y is 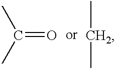
and R¹⁸ is hydrogen, lower alkyl or lower aralkyl, or, when p is 1, the amide bond between D and E being optionally substituted by 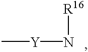
wherein Y and R¹⁸ are as indicated above;
or a pharmaceutically acceptable salt thereof, together with a pharmaceutically acceptable carrier or diluent.

2. A compound according to claim 1, wherein
p is 1,
A is hydrogen or R¹—(CH₂)_{q}—(X)ᵣ—(CH₂)ₛ—CO—,
wherein R¹ is 3-imidazolyl, q is 2, r is 0 and s is 0;
or wherein R¹ is NH₂, q is 1, r is 1, X is disubstituted benzene preferably substituted in the 1 and 3 positions, and s is 0;
or wherein R¹ is NH, q is 1, r is 1, X is disubstituted thiophene preferably substituted in the 3 and 2 positions, and s is 0;
t is 1;
G is Ala, Gly, Aib, sarcosine, nipecotic acid, or iso-nipecotic acid;
u is 1;
H is His, Phe, Tic, 3Pyal, Gly, Ala, Phe(4-NH₂), Sar, Pro, Tyr, Arg, Orn, 3-aminomethylbenzoic acid or D-Phe;
R⁴ is 2-naphthyl;
R⁵ is phenyl;
v is 2, 3, 4, 5, or 6;
R⁹ is —NH₂, morpholinopropyl, morphoninoethyl or (1-methylpyrrolidinyl)ethyl; and
R²⁰ is —COOH, —CH₂—OH, —H or —CONH₂.

3. A composition according to claims 2 or 3 wherein the compound of general formula I is selected from the group consisting of
H-Ala-Hisψ(CH₂NH)D-2Nal-D-Phe-Lys-NH₂,
H-Ala-Ala-D-2Nal-D-Phe-Lys-NH₂,
H-His-D-2Nal-D-Phe-Lys-NH₂,
(3-(4-imidazolyl)propionyl)-D-2Nal-D-Phe-Lys-NH₂,
H-D-Lys-D-2Nal-D-Phe-Lys-NH₂,
H-5Apent-His-D-2Nal-D-Phe-Lys-NH₂,
H-D-Ala-D-2Nal-D-Phe-Lys-NH₂,
H-5Apent-D-2Nal-D-Phe-Lys-NH₂,
(n-Propyl)-His-D-2Nal-D-Phe-Lys-NH₂,
H-Ala-3Pyal-D-2Nal-D-Phe-Lys-NH₂,
H-Ala-Phe(4-NH₂)-D-2Nal-D-Phe-Lys-NH₂,
H-D-Ala-His-D-2Nal-D-Phe-Lys-NH₂,
(2-(4-imidazolyl)acetyl)-D-2Nal-D-Phe-Lys-NH₂,
(3-(4-imidazolyl)acryloyl)-D-2Nal-D-Phe-Lys-NH₂,
(3-Aminomethylbenzoyl)-D-2Nal-D-Phe-Lys-NH₂,
(3-Aminophenylacetyl)-D-2Nal-D-Phe-Lys-NH₂,
(4-Aminophenylacetyl)-D-2Nal-D-Phe-Lys-NH₂,
(3-Aminocrotonoyl)-D-2Nal-D-Phe-Lys-NH₂,
(4-Piperidino-carboxyl)-D-2Nal-D-Phe-Lys-NH₂,
H-Ala-His-D-2Nal-D-Phe-NH₂,
(H-Ala-His-D-2Nal-D-Phe-NH)hexane,
6-(H-Ala-His-D-2Nal-D-Phe-NH)hexylamine,
5-(H-Ala-His-D-2Nal-D-Phe-NH)pentylanaine,
H-Ala-His-D-2Nal-D-Pheψ(CH₂NH)Lys-NH₂,
H-Ala-His-D-2Nal-D-Phe-Lys-OH,
(2S)-(H-Ala-His-D-2Nal-D-Phe-NH)-6-aminohexanol,
(2-(H-Ala-His-D-2Nal-D-Phe-NH)ethyl)benzene,
2-(H-Ala-His-D-2Nal-D-Phe-NH)ethylamine,
4-((H-Ala-His-D-2Nal-D-Phe-NH)methyl)benzylamine,
H-Ala-His-D-2Nal-D-Phe-Lys(maltosyl)-NH₂,
H-Ala-His-D-2Nal-D-Phe-Phe-NH₂,
H-Ala-His-D-2Nal-D-Phe-D-Phe-NH₂,
H-Ala-His-D-Phe-D-Phe-Lys-NH₂,
H-Ala-His-D-Trp-D-Phe-Lys-NH₂,
H-His-D-2Nal-D-Trp-Lys-NH₂,
H-Ala-His-D-1Nal-D-Phe-Lys-NH₂,
H-Ala-Phe-D-2Nal-D-Phe-Lys-NH₂,
H-Ala-His-D-2Nal-D-Phe-Lys(maltosyl)-NH₂,
(2R)-(H-Ala-His-D-2Nal-D-Phe-Lys-NH)-3-phenylpropylamine,
H-Ala-N-Me-(2-aminobenzoyl)-D-2Nal-D-Phe-Lys-NH₂,
(3-(Methylaminomethyl)benzoyl)-D-2Nal-D-Phe-Lys-NH₂,
(4-(Aminomethyl)benzoyl)-D-2Nal-D-Phe-Lys-NH₂,
H-His-Ala-D-2Nal-D-Phe-Lys-NH₂,
4-(H-Ala-His-D-2Nal-D-Phe-NH)butylamine,
3-(H-Ala-His-D-2Nal-D-Phe-NH)propylamine,
(3-(Dimethylaminomethyl)benzoyl)-D-2Nal-D-Phe-Lys-NH₂,
(3-Amino-3-methylbutanoyl)-D-2Nal-D-Phe-Lys-NH₂,
(3-Aminomethylbenzoyl)-D-hPhe-D-Phe-Lys-NH₂,
(3-Aminomethylbenzoyl)ψ(CH₂NH)D-2Nal-D-Phe-Lys-NH₂,
(3-Aminomethylbenzoyl)-D-2Nal-D-hPhe-Lys-NH₂,
(3-Amino-3-methylbutanoyl)-His-D-2Nal-D-Phe-Lys-NH₂,
(3-Aminomethylbenzoyl)-D-2Nal-N-Bzl-Gly-Lys-NH₂,
(2S)-(3-aminomethylbenzoyl)ψ(CH₂NH)-D-2Nal-D-Phe-NH)-6-aminohexanol,
(2S)-((3-aminomethylbenzoyl)-D-2Nal-D-Phe-NH)-6-aminohexanol,
(3-Aminomethylbenzoyl)-D-2Nal-D-Thial-Lys-NH₂,
(2S)-(H-Aib-Hisψ(CH₂NH)-D-2Nal-D-Phe-NH)-6-aminohexanol,
(3-Aminomethylbenzoyl)-D-2Nal-D-3Pyal-Lys-NH₂,
(3-Aminomethylbenzoyl)-D-2Nal-D-Phe(4-F)-Lys-NH₂,
(3-Aminomethylbenzoyl)-D-2Nal-D-Phe(4-OMe)-Lys-NH₂,
(2-Aminomethylphenylacetyl)-D-2Na(-D-Phe-Lys-NH₂,
(2-Aminomethylbenzoyl)-D-2Nal-D-Phe-Lys-NH₂,
2-(H-Aib-His-D-2Nal-D-Phe-NH)-(4pyridyl)ethane,
H-Aib-Phe-D-2Nal-D-Phe-Lys-NH₂,
2-(H-Aib-His-D-2Nal-D-Phe-NH)-(1-methyl-2-pyrrolidinyl)ethane,
2-(H-Aib-His-D-2Nal-D-Phe-NH)-(4pyrldyl)ethane,
H-Aib-Hisψ(CH₂NH)-D-2Nal-D-Phe-Lys-OH,
(3-Aminomethylbenzoyl)-D-2Nal-N-Me-D-Phe-Lys-NH₂,
H-Aib-His-D-2Nal-D-Phe-Gly-NH₂,
H-Aib-His-D-2Nal-D-Phe-Ala-NH₂,
H-Aib-His-D-2Nal-D-Phe-Orn-NH₂,
(5-Aminomethylthienyl-2-carbonyl)-D-2Nal-D-Phe-Lys-NH₂,
H-Aib-His-D-2Nal-D-Phe-D-Lys-NH₂,
H-Aib-His-D-2Nal-D-Phe-Dab-NH₂,
H-Aib-His-D-2Nal-D-Pheψ(CH₂NH)-Lys-NH₂,
H-Aib-His-N-Me-D-2Nal-D-Phe-Lys-NH₂,
H-Aib-His-D-2Nal-D-Phe-N-Me-Lys-NH₂,
(3-Aminomethylthienyl-2-carbonyl)-D-2Nal-D-Phe-Lys-NH₂,
H-Aib-His-D-2Nal-N-Me-D-Phe-Lys-NH₂,
H-Aib-His-D-2Nal-D-Phe-Lys-N(Me)₂,
(3R)-Piperidinecarbonyl-D-2Nal-D-Phe-Lys-NH₂,
(3S)-Piperidinecarbonyl-D-2Nal-D-Phe-Lys-NH₂,
(3-Aminomethylbenzoyl)-D-1Nal-D-Phe-Lys-NH₂,
H-Aib-His-D-2Nal-D-Trp-Lys-NH₂,
(Furfuryl)-Aib-His-D-2Nal-D-Phe-Lys-NH₂,
(2-Pyridylmethyl)-Aib-His-D-2Nal-D-Phe-Lys-NH₂,
H-Aib-(3-aminomethylbenzoyl)-D-2Nal-D-Phe-Lys-NH₂,
H-Aib-3Pyal-D-2Nal-D-Phe-Lys-NH₂,
(3S)-Piperidinecarbonyl-D-2Nal-D-Phe-Lys-NH₂,
(3R)-Piperidinecarbonyl-D-2Nal-D-Phe-Lys-NH₂,
(2-(H-Aib-His-D-2Nal-NH)ethyl)benzene,
N,N-di(2R-Hydroxypropyl)-(3-aminomethylbenzoyl)-D-2Nal-D-Phe-Lys-NH₂,
(2R-Hydroxypropyl)-Aib-His-D-2Nal-D-Phe-Lys-NH₂,
(3-Aminomethylbenzoyl)-D-2Nal-D-Pheψ(CH₂NH)Lys-NH₂,
(3-Aminomethylbenzoyl)-N-Me-D-2Nal-D-Phe-Lys-NH₂,
(3-Aminomethylbenzoyl)-D-2Nal-D-Phe-N-Me-Lys-NH₂,
H-D-Thr-His-D-2Nal-D-Phe-Lys-NH₂,
H-Aib-His-D-2Nal-N-(phenethyl)-Gly-Lys-NH₂,
(3-Aminomethylbenzoyl)-D-2Nal-N-(phenethyl)-Gly-Lys-NH₂,
H-Hyp-His-D-2Nal-D-Phe-Lys-NH₂,
H-Aib-His-N-Me-D-2Nal-N-(phenethyl)-Gly-Lys-NH₂,
H-Aib-His-N-Me-D-2Nal-N-Me-D-Phe-Lys-NH₂,
H-Aib-His-D-2Nal-D-Pheψ(CH₂N(Me))Lys-NH₂,
3-(H-Aib-His-D-2Nal-N-Me-D-Phe-NH)morpholinopropane,
2-(H-Aib-His-D-2Nal-N-Me-D-Phe-NH)-(1-methyl-2-pyrrolidinyl)ethane,
(3R)-Piperidinecarbonyl-N-Me-D-2Nal-N-Me-D-Phe-Lys-NH₂,
3-((Aminomethylbenzoyl)-D-2Nal-N-Me-D-Phe-NH)morpholinopropane,
2-(H-Aib-His-N-Me-D-2Nal-N-Me-D-Phe-NH)-(1-methyl-2-pyrrolidinyl)ethane,
2-(3R)-Piperdinecarbonyl-N-Me-D-2Nal-N-Me-D-Phe-NH)-(1-methyl-2-pyrrolidinyl)ethane,
2-(3-Aminomethylbenzoyl)-N-Me-D-2Nal-N-Me-D-Phe-NH)-(1-methyl-2-pyrrolidinyl)ethane,
3-(H-Aib-His-N-Me-D-2Nal-N-Me-D-Phe-NH)morpholinopropane,
3-((3R)-Piperidinecarbonyl-N-Me-D-2Nal-N-Me-D-Phe-NH)morpholinopropane,
3-((3-Aminomethylbenzoyl)-N-Me-D-2Nal-N-Me-D-Phe-NH)morpholinopropane,
H-Aib-His-D-2Nal-N-Me-D-Phe-Hyp-NH₂,
2-((3-Aminomethylbenzoyl)-D-2Nal-N-Me-D-Phe-NH)-(1-methyl-2-pyrrolidinyl)ethane,
2-((3R)Piperidinecarbonyl-D-2Nal-N-Me-D-Phe-NH)-(1-methyl-2-pyrrolidinyl)ethane,
ipamorelin (H-Aib-His-D-2Nal-D-Phe-Lys-NH₂), and
pharmaceutically acceptable salts thereof.

4. A pharmaceutical composition comprising a compound of the general formula I or a pharmaceutically acceptable salt thereof and a TRH analogue, together with a pharmaceutically acceptable carrier or diluent.

5. A composition according to any one of the claims 1-4 wherein the compound of the general formula I is ipamorelin or a pharmaceutically acceptable salt thereof.

6. A method for the treatment of the catabolic state of prolonged critical illness, the method comprising administering to a subject in need thereof an effective amount of a compound of the general formula I or a pharmaceutically acceptable salt thereof and an effective amount of TRH.

7. A method for the treatment of the catabolic state of prolonged critical illness, the method comprising administering to a subject in need thereof an effective amount of a compound of the general formula I or a pharmaceutically acceptable salt thereof and an effective amount of a TRH analogue.

8. A method according to the claims 6 or 7, wherein the compound of the general formula I is ipamorelin or a pharmaceutically acceptable salt thereof.

9. Use of a compound of the general formula I or a pharmaceutically acceptable salt thereof together with TRH for the preparation of a medicament for the treatment of the catabolic state of prolonged critical illness.

10. Use of a compound of the general formula I or a pharmaceutically acceptable salt thereof together with a TRH analogue for the preparation of a medicament for the treatment of the catabolic state of prolonged critical illness.

11. The use according to the claims 9 or 10 wherein the compound according to the general formula I is parnorelin or a pharmaceutically acceptable salt thereof.

12. A kit comprising a compound of the general formula I or a pharmaceutically acceptable salt thereof and TRH

13. A kit comprising a compound of the general formula I or a pharmaceutically acceptable salt thereof and a TRH analogue.

14. The kit according to the claims 12 or 13 wherein the compound according to the general formula I is ipamorelin or a pharmaceutically acceptable salt thereof.

15. Use of ipamorelin or a pharmaceutically acceptable salt thereof for the preparation of a medicament for use in the treatment of the catabolic state of prolonged critical illness in a regimen which additionally comprises treatment with TRH.

16. Use of ipamorelin or a pharmaceutically acceptable salt thereof for the preparation of a medicament for use in the treatment of the catabolic state of prolonged critical illness in a regimen which additionally comprises treatment with a TRH analogue.

17. The use according to the claims 15 or 16 wherein the compound according to the general formula I is ipamorelin or a pharmaceutically acceptable salt thereof.
